(19) United States
(12) Patent Application Publication
(10) Pub. No.: US 2012/0089173 A1
(43) Pub. Date: Apr. 12, 2012

(54) FILTER WITH MAGNETIC TIP FOR CLOT FRAGMENTATION

(75) Inventor: Kurt J. Tekulve, Ellettsville, IN (US)

(73) Assignee: Cook Incorporated, Bloomington, IN (US)

(21) Appl. No.: 12/899,665

(22) Filed: Oct. 7, 2010

(51) Int. Cl. A61F 2/01

(52) U.S. Cl. 606/200

(57) ABSTRACT

A magnetically polarizable, filtering device system and a method of using such a system for capturing embolic material contained within fluid flowing through a body vessel of a patient is provided. The filtering device system generally comprises a source adapted to generate a magnetic field and a filter device adapted to capture embolic material and to unclog itself when necessary or desirable.

### FIELD

This disclosure relates generally to medical devices. More specifically, this disclosure relates to a filter device and the use of such a device to capture and remove embolic material from a body lumen or vessel.

### BACKGROUND

Filter devices that are percutaneously placed into a body lumen or vessel, such as the vena cava, are routinely used with orthopedic surgery patients, neurosurgery patients, or in patients having medical conditions that require substantial bed rest. During such medical conditions, the need for these filtering devices arises due to the likelihood that blood clots will form in the peripheral vasculatures of the patient. These blood clots can easily break away from the vessel wall, thereby, creating the risk of downstream embolism. The occurrence of an embolism is a life-threatening condition due to the possibility that the clot will come to rest in a location that obstructs the flow of blood through the body lumen, thereby, cutting off the body's supply of oxygen.

Although filtering devices are widely accepted and used, the existing filter configurations suffer from a variety of shortcomings. For example, such filters are highly susceptible to becoming clogged with embolic material. When a filter becomes clogged with embolic material, the flow of blood through the lumen is substantially reduced. When this occurs, the patient must be further treated to restore the flow of blood through the body lumen.

### SUMMARY

In overcoming the enumerated drawbacks and other limitations of the related art, the present disclosure provides a magnetically polarizable, filtering device system and a method of using such a system for capturing embolic material contained within fluid flowing through a body vessel of a patient. The filtering device system generally comprises a source adapted to generate a magnetic field and a filter device. The filter device being adapted to capture embolic material and to unclog itself when necessary or desirable.

The filter device, constructed in accordance with the teachings of the present disclosure, generally comprises a hub, a set of wires, and at least one core wire having a magnetically polarizable portion. The set of wires and core wire are coupled to and extend from the hub. The set of wires further includes a plurality of struts that have a predetermined shape and are configured to move between an expanded state for engagement with the body vessel and a collapsed state for filter retrieval and delivery. These struts define an internal volume in the device's expanded state in which the embolic material is captured. The application of a magnetic field arising from the source induces the core wire to vibrate and break-up the captured embolic material in order to maintain the flow of fluid through the body vessel.

According to another aspect of the present disclosure, the filter device may be percutaneously delivered and retrieved from the body vessel. The source of the magnetic field is located external to the body vessel, preferably, external to the patient.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is an illustration of the anatomy of the vena cava in which a filter device configured according to the teachings of the present disclosure is deployed;

FIG. 2 is a side perspective view of a filter device according to one aspect of the present disclosure;

FIG. 3A is a cross-sectional view of the vena cava depicting the filter device of FIG. 2 partially deployed in a manner in which the removal hook leads the way;

FIG. 3B is a cross-sectional view of the vena cava depicting the filter device of FIG. 2 partially deployed in a manner in which the second end of the struts leads the way;

FIG. 4A is a cross-sectional view of a body vessel depicting a filtering device system including the filter device of FIG. 2 fully deployed to collect embolic material;

FIG. 4B is a cross-sectional view of a body vessel depicting a filtering device system including the filter of FIG. 4A in which the core wire is used to fragment or break-up the collected embolic material;

FIG. 5A is a cross-sectional view of a body vessel in which a retrieval sheath engages the primary struts of the filter device in FIG. 4B; and

FIG. 5B is a cross-sectional view of a body vessel in which the retrieval sheath includes the filter device of FIG. 4B in the collapsed state for removal.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

The present disclosure generally provides a filter device and a method of using such a device to capture and remove embolic material from a body lumen. As a result, the filter device of the present disclosure may be used to improve the circulation of blood through the body lumen and to reduce the chance of clot related issues, such as a stroke or pulmonary embolism.

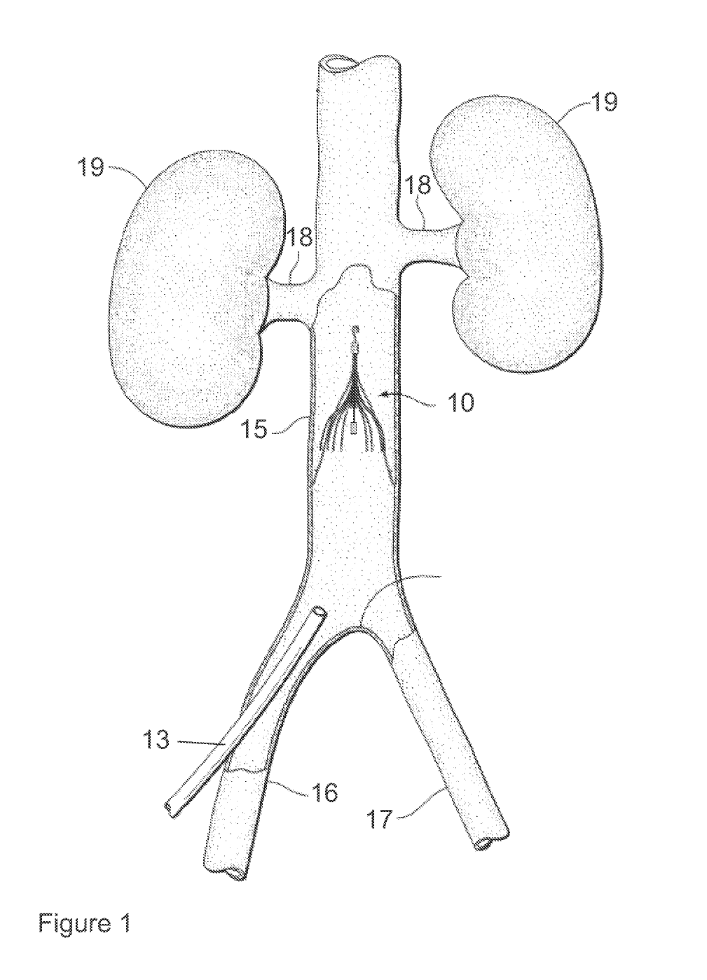

Referring to FIG. 1, a filter device 10 constructed according to the teachings of the present disclosure is illustrated, for example, as implanted in the inferior vena cava 15 of a patient. The filtering device 10 may be delivered to such a treatment location through the use of a delivery tube or catheter 13. The filter device 10 includes features that lessen the likelihood of perforating the wall of the vena cava and that will allow the filter to be removed when desirable. In this example, the filter device 10 is positioned to capture embolic material or thrombi carried by the blood flowing through the iliac veins 16, 17 into the pulmonary arteries and toward the renal veins 18 from the kidneys 19 and toward the patient's heart.

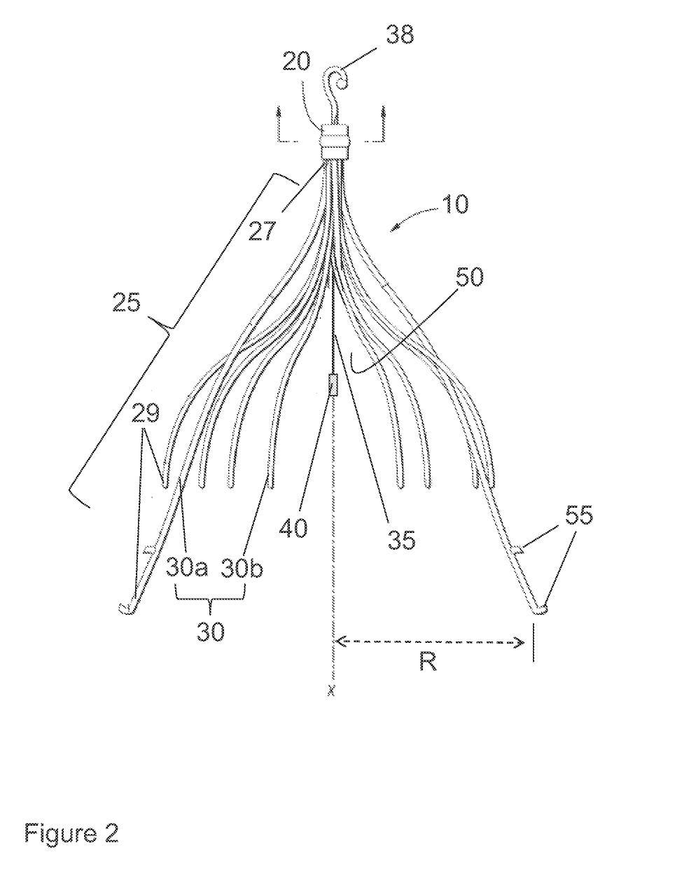

Referring now to FIG. 2, the filter device generally comprises a hub 20, a set of wires 25 including a plurality of struts 30, and at least one core wire 35. Each of the struts 30 and the core wire 35 includes a first end 27 and a second end 29. The first end 27 of each strut 30 and core wire 35 are coupled to and extend from the hub 20. Each of the struts 30 has a predetermined shape and is configured to move between an expanded state for engagement with the blood vessel and a collapsed state for filter retrieval and delivery. The core wire 35 includes a magnetically polarizable portion or tip 40 that is proximate to its second end 29. The hub 20 is coupled to the first end 27 of the struts 30 and core wire 35 by any means known to one skilled-in-the-art, including but not limited to crimping. The attachment of the hub 20 causes the first end 27 of the struts 30 and core wire 35 to form a compact bundle centered about the longitudinal axis (x) of the filter device 10. The minimal diameter of the hub 20 is predetermined by the size of the struts 30 and core wire 35 used to form the set of wires 25.

According to one aspect of the disclosure, the filter device 10 may include two layers or portions of struts 30, namely, primary struts 30a and secondary struts 30b with the struts 30 longitudinally engaging the vessel wall of the body lumen. One skilled-in-the-art will understand that the filter device 10 may include only one layer of struts or more than two layers of struts without exceeding the scope of this disclosure. The length of the filter device 10 is preferably defined by the length of its primary struts 30a. Furthermore, the diameter of the hub 20 is generally defined by the size of a bundle that contains the first ends 27 of the primary struts 30a, secondary struts 30b, and core wire 35. In the example shown in FIG. 2, a filter device 10 having eight secondary struts 30b and two primary struts 30a is shown. However, the number of struts 30 can be greater than or less than that depicted in FIG. 2 if desired or necessary as determined for the selected application. The filter device 10 may further include a removal hook 38 that extends from hub 20 opposite the first ends 27 of the primary 30a struts, secondary struts 30b, and core wire 35.

Still referring to FIG. 2, according to another aspect of the present disclosure, the struts 30 may be configured to provide the body of the filter device 10 with a substantially conical shape. In an expanded state, the shape of the filter 10 will define an interior volume 50 that provides an entrapment region for capturing and holding embolic material. The spacing between the struts 30 can be predetermined and configured for use in a given application. However, in one preferred configuration, the struts are spaced for capturing clots that have a size of about 7 mm or greater, with smaller particles being allowed to pass there through. The struts 30 are preferably arranged to create very little resistance to blood flow through the body lumen. The filter device 10 is preferably configured to be collapsible into a smaller cross-sectional profile for facilitating its delivery to and retrieval from the targeted treatment site.

One or more anchors 55 may optionally be provided along the second ends 29 of at least one of the struts 30 for use in engaging the inner wall of the body vessel. These anchors may comprise barbs, hooks, or any other shape well-suited for engagement with the inner wall. Preferably, the anchors are sized and adapted such that they cannot penetrate through the wall of the blood vessel. During the operational life-time of the filter device 10, the anchors 55 may become incorporated into the endothelial tissue associated with the inner wall of the body vessel, thereby, reducing the possibility of any undesirable migration of the filter. According to yet another variation of the present disclosure, the filter device 10 may be supported by an expandable stent structure (not shown) that expands for engagement with the inner wall of the vessel. The stent may also be used to reduce the chance of undesirable filter migration.

Preferably, the struts 30 are formed of a superelastic material, stainless steel wire, Nitinol, cobalt-chromium-nickel-molybdenum-iron alloy, or cobalt chrome-alloy or any other material suitable for use in a filter device that allows for the transitioning between an expanded state and a collapsed state. According to one aspect of the present disclosure, the struts 30 are preferably formed from wire having a round cross-section with a diameter of at least about 0.35 mm. Of course, it is not necessary that the struts 30 have a round or near round cross-section. For example, the struts 30 could take on any shape with rounded edges to maintain non-turbulent blood flow through the interior volume 50 of the filter device 10.

The struts 30 are configured to move between an expanded state for engagement with the body vessel and a collapsed state for retrieval or delivery of the filter device 10 into the body vessel. In the expanded state, each of the struts 30 extends arcuately along a longitudinal axis (x) and linearly relative to a radial axis (R) (see FIG. 2) from its first end 27 to the second end 29. The radial axis (R) is defined by the struts 30 as they radially extend from their first ends 27 to their second ends 29.

The core wire 35 is an elongate member also having a first end 27 and a second end 29 with a magnetically polarizable portion or tip 40 located proximate to the second end 29. The first end 27 is coupled to the hub 20. The core wire 35 is preferably disposed within the interior volume 50 of the filter device 10. The magnetically polarizable tip 40 is adapted for engaging the captured embolic material. The core wire 35 and its tip 40 is configured to break apart a clot of embolic material by producing forces that assist or facilitate breaking the embolus into smaller pieces. These smaller pieces can be more easily emulsified by the body's natural lytic system. The core wire 35 may have a cross-sectional profile similar to or smaller than that of the struts 30 of the filter device 10.

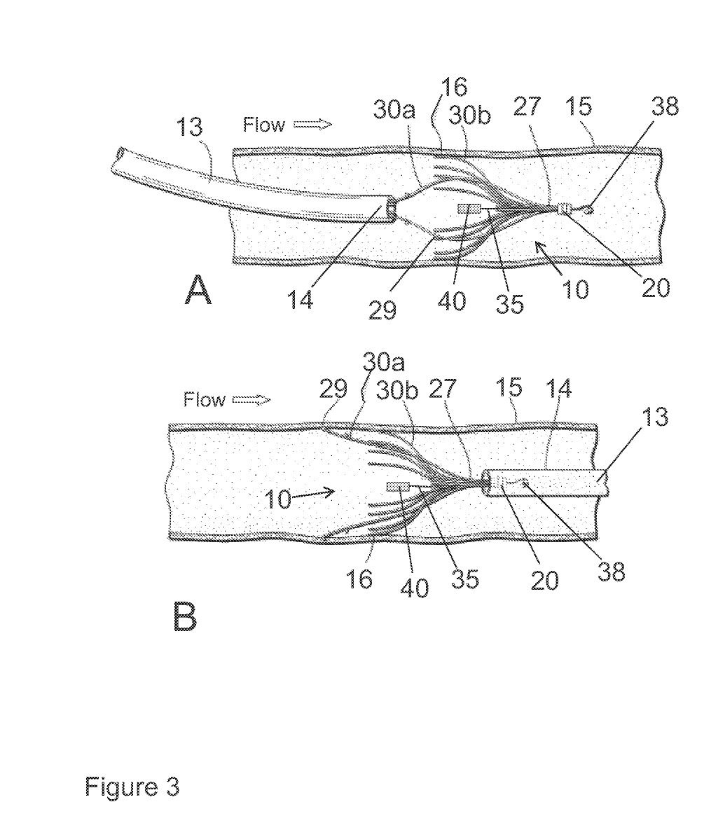

Referring now to FIGS. 3A and 3B, the filter device 10 partially deployed in the inferior vena cava 15 of a patient is shown. In FIG. 3A the filter device 10 is being delivered into the vena cava 15 by a delivery tube 13 percutaneously through the vasculature (not shown) of a patient. In this case, the filter device 10 is inserted through the proximal end of the delivery tube 13 with the removal hook 38 leading and the second ends 29 of the struts 30 being held by a filter retainer member for delivery via the femoral vein of a patient.

FIG. 3B shows a similar filter device 10 being delivered by a delivery tube 13 percutaneously through the jugular vein (not shown) of a patient. In each case (FIGS. 3A and 3B) the distal end 14 of the delivery tube 13 is positioned by a physician proximate to the targeted or desired location for deployment. In this case, the filter device 10 is inserted through the proximal end of the delivery tube 13 with the second ends 29 of the struts 30 leading and the removal hook 38 trailing for delivery via the jugular vein of a patient. According to this aspect of the present disclosure, a pusher wire (not shown) having a pusher member at its distal end may be fed through the proximal end of the delivery tube 13, thereby, pushing the filter device 10 through the tube until it reaches the distal end 14 of the tube 13 that is positioned proximate to the targeted location.

Still referring to FIGS. 3A and 3B, during the deployment of the filter device 10, the secondary struts 30b expand first to centralize or balance the filter within the body vessel. When the second ends 29 of the primary 30a and secondary struts 30b emerge from the distal end of the delivery tube 13, the struts 30 expand to an expanded position with the second ends 29 of the struts 30 engaging the inner wall 16 of the vessel 15. The engagement of the struts 30 with the inner wall 16 of the vessel 15 anchors the filter device 10 at the location of deployment in the vessel 15, thereby, preventing the filter device 10 from being moved by the blood flowing through the vessel 15.

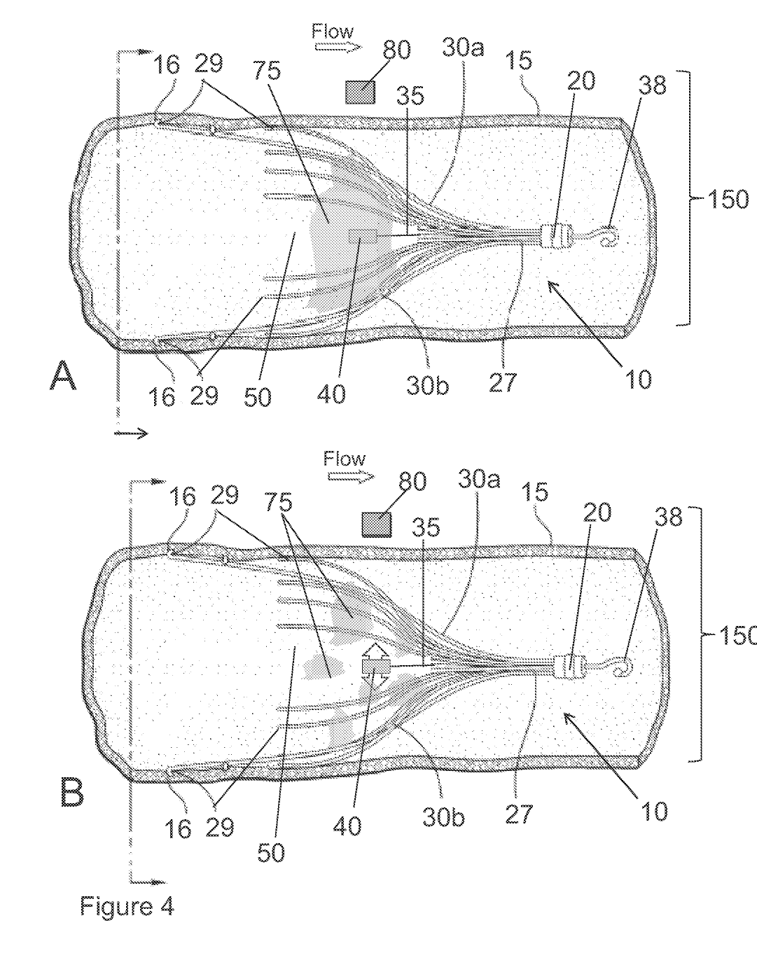

Referring now to FIG. 4A, when embolic material 75 (or other particles present in the blood) reaches the filter device 10, the embolic material 75 enters the mouth of the filter device 10 and is funneled toward the center of the interior volume 50. When captured by the struts 30 of the filter device 10, the embolic material 75 remains lodged in the filter device 10. As more and more embolic material 75 is captured, the filter device 10 may become plugged, thereby, decreasing the flow of blood through the vessel 15.

Referring now to FIG. 4B, in order to increase the flow of blood through the vessel 15, the mass of accumulated embolic material 75 needs to be fragmented into smaller masses or pieces 76. The fragmentation of the embolic material 75 into smaller pieces 76 may be accomplished using the magnetically polarizable portion or tip 40 of the core wire 35. In this case, the placement of a magnetic source 80 adapted to apply a magnetic field in the vicinity of the magnetically polarizable portion or tip 40 will cause the tip 40 to polarize and move in the direction of the magnetic field source 80. The combination of at least one magnetic source 80 and the aforementioned filter device 10 comprises a magnetically polarizable, filtering device system 150 for use by a physician.

Preferably, the magnetic field is applied perpendicular to the core wire 35, however, any angle offset from the longitudinal axis (x) of the filter device 10 will suffice. Alternating the magnetic field by either turning the field on and off or via the direct application of an alternating magnetic field, the attached core wire 35 with its polarized tip 40 will begin to move or vibrate. The magnetic field may be generated through the use of at least one magnetic source 80 positioned external to the body lumen, preferably, external to the patient.

The movement or vibration of the magnetically polarizable portion or tip 40 and core wire 35 provides the forces required to fragment, divide, or break-up the embolic material 75 into smaller, remaining pieces 76. As the embolic material 75 is broken into smaller and smaller pieces 76, the body's lytic capabilities are able to more quickly dissolve some of the smallest pieces 76. The larger of the remaining pieces 76 may continue to be held within the interior volume 50 of the filter device 10 or depending upon the configuration of the filter device 10 some of the harmless smaller pieces 76 may be able to pass through the struts 30 of the filter device 10. The smaller remaining pieces 76 that pass through the struts 30 will be dissolved by the body's lytic capabilities downstream from the filter device 10.

In order to further enhance the dissolution of embolic material 75, one or more thrombolytic drugs or therapeutic agents may be used in addition to the filter device 10. The various components of the filter device 10 may, if desirable, be coated with one or more of such drugs. The therapeutic agents may include, but not be limited to, antiproliferative agents, anti-inflammatory agents, and antiplatelet agents, among others.

The magnetically polarizable portion or tip 40 of the core wire 35 may be made from any ferromagnetic, ferromagnetic, paramagnetic, or superparamagnetic material or mixtures thereof that are known to one skilled-in-the-art. Examples, of such materials include, but are not limited to, iron, iron oxide, iron nitride, iron carbide, chromium dioxide, silicon steel, nickel, nickel alloys, cobalt, iron/cobalt alloys, magnetic stainless steels, and ferrites, among others. These materials may further include any known alloys of iron, such as those containing aluminum, silicon, cobalt, nickel, vanadium, molybdenum, chromium, tungsten, manganese, and/or copper. The magnetically polarizable tip 40 may be coated by or encapsulated within a bio-compatible material in order to ensure there is no detrimental affects upon exposure to body fluids.

The magnetic field used to polarize the tip 40 of the core wire 35, may be generated using a permanent magnet, electromagnet, or a combination thereof as the source 80. Such a source 80 may be positioned external to the body lumen in which the filter device 10 is deployed or, preferably, external to the patient. Although the use of one source 80 is preferred, one skilled-in-the-art will understand that multiple sources 80 may be utilized without exceeding the scope of the present disclosure. The controllability of the magnetic field strength can be accomplished by varying the amount and direction of electric current flowing through the electromagnet or the orientation of the permanent magnets. The strength or magnitude of the magnetic field applied is predetermined to generate sufficient movement of the magnetically polarizable tip 40 to transfer at least the minimum forces necessary to cause fragmentation of the embolic material 75.

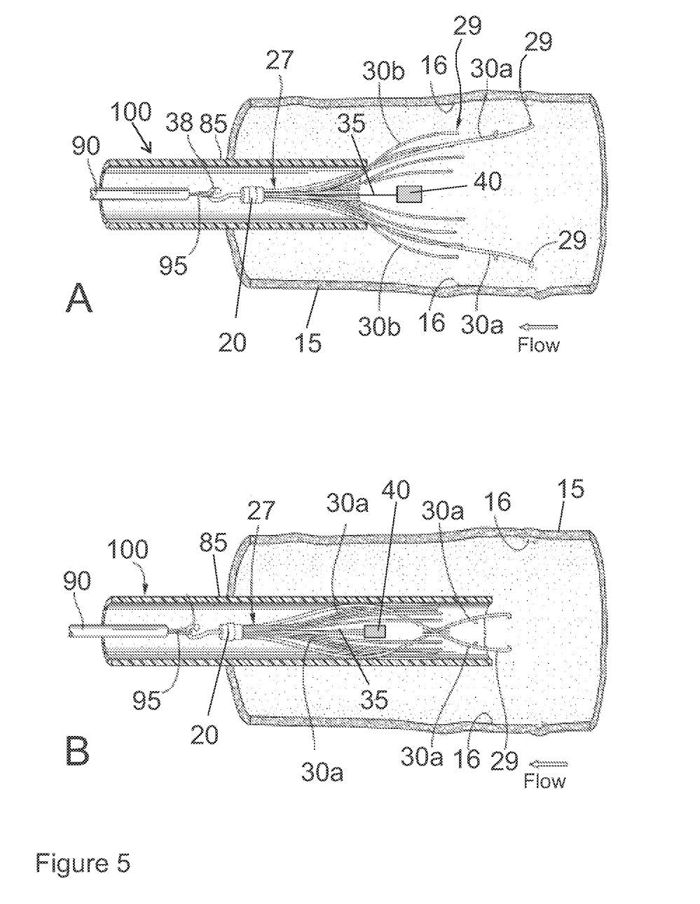

Referring now to FIGS. 5A and 5B, when the filter device 10 is to be removed, the removal hook 38 is preferably grasped by a retrieval instrument 100 that is percutaneously introduced in the vena cava 15 in the direction of removal hook 38. In this procedure (see FIG. 5A), a removal catheter, sheath, or tube 85 of the retrieval device 100 is inserted into the superior vena cava 15. A wire 90 having a loop snare 95 at its distal end or another means to interact with the removal hook 38 of the filter device is threaded through the removal sheath 85 and exits through the sheath's distal end. The wire 90 is then manipulated by any suitable means from the proximal end of the retrieval device such that the loop snare 95 captures the removal hook 38 of the filter device 10. Using counter traction by pulling the wire 90 while pushing the sheath 85, the sheath 85 is passed over the filter device 10.

Referring now to FIG. 5B, as the sheath 85 passes over the filter device 10, the struts 30 (including both primary struts 30a and secondary struts 30b) engage the edge of the sheath 85 and are forced to pivot or undergo bend deflection at the hub 20 toward the longitudinal axis (x) of the filter device 10. The pivot motion toward the longitudinal axis (x) causes the second ends 29 of the struts 30 to be retracted from the inner surface of the vessel wall 16. Only surface lesions and small point lesions on the vessel wall are created during this removal procedure. One skilled-in-the-art will understand that any other suitable procedure may be implemented to remove the filter device 10 from the patient.

A person skilled in the art will recognize that the measurements described are standard measurements that can be obtained by a variety of different test methods. The test methods described in the examples represents only one available method to obtain each of the required measurements.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A filter device for capturing embolic material contained within fluid flowing through a body vessel, the filter device comprising:
a hub;
a set of wires coupled to and extending from the hub, the set of wires including a plurality of struts having a predetermined shape and being configured to move between an expanded state for engagement with the body vessel and a collapsed state for filter retrieval and delivery; the struts defining an internal volume in the expanded state in which the embolic material is captured; and
at least one core wire a portion of which is magnetically polarizable; the core wire being coupled to the hub such that the magnetically polarizable portion extends into the internal volume defined by the struts;
wherein the application of an external, variable magnetic field induces the core wire to vibrate and break-up the captured embolic material in order to maintain the flow of fluid through the body vessel.

2. The filter device of claim 1, wherein the filter device has a longitudinal axis (x) with the attachment of the hub to the struts and the core wire forming a compact bundle centered about this axis (x).

3. The filter device of claim 1, wherein the struts include two layers; one layer being primary struts; the second layer being secondary struts; the primary struts defining the length of the filter device.

4. The filter device of claim 1, wherein at least one strut includes at least one anchor adapted to engage the body vessel.

5. The filter device of claim 1, wherein the struts are made from a material selected as one from the group of a superelastic material, stainless steel wire, Nitinol, cobalt-chromium-nickel-molybdenum-iron alloy, and cobalt chrome-alloy.

6. The filter device of claim 1, wherein the core wire is further defined by a first end and a second end, the first end being coupled to the hub, and the magnetically polarizable portion of the core wire being proximate to the second end.

7. The filter device of claim 1, wherein the core wire has a cross-sectional profile substantially similar to or smaller than that of the struts.

8. The filter device of claim 1, wherein the filter device further comprises a retrieval hook coupled to the hub.

9. The filter device of claim 1, wherein at least one strut is coated with one or more thrombolytic drugs or therapeutic agents.

10. The filter device of claim 6, wherein the magnetically polarizable portion is made from one selected from the group of a ferromagnetic, ferromagnetic, paramagnetic, and superparamagnetic material or mixture thereof.

11. The filter device of claim 10, wherein the magnetically polarizable portion is made from one selected from the group of iron, iron oxide, iron nitride, iron carbide, chromium dioxide, silicon steel, nickel, nickel alloys, cobalt, iron/cobalt alloys, magnetic stainless steels, and ferrites.

12. The filter device of claim 6, wherein the magnetically polarizable portion is coated by or encapsulated within a bio-compatible material.

13. A magnetically polarizable, filtering device system for use in capturing embolic material contained within fluid flowing through a body vessel, the filtering device system comprising:
at least one source for generating a magnetic field; and
a filter device; the filter device including:
a hub;
a set of wires coupled to and extending from the hub, the set of wires including a plurality of struts having a predetermined shape and being configured to move between an expanded state for engagement with the body vessel and a collapsed state for filter retrieval and delivery; the struts defining an internal volume in the expanded state in which the embolic material is captured; and
at least one core wire a portion of which is magnetically polarizable; the core wire being coupled to the hub such that the magnetically polarizable portion extends into the internal volume defined by the struts;
wherein the application of the magnetic field induces the core wire to vibrate and break-up the captured embolic material in order to maintain the flow of fluid through the body vessel.

14. The filtering device system of claim 13, wherein the source generates a magnetic field through the use of one selected from the group of a permanent magnet, electromagnet, and a combination thereof.

15. The filtering device system of claim 14, wherein the source is positioned external to the body vessel in which the filter device is deployed or is external to the patient.

16. The filtering device system of claim 14, wherein the magnetic field is a variable magnetic field generated by turning the field on and off or by the direct application of an alternating magnetic field.

17. The filtering device system of claim 14, wherein the filter device further includes a longitudinal axis (x) and the magnetic field is applied to the core wire at an angle that is offset from the longitudinal axis (x).

18. The filtering device system of claim 17, wherein the magnetic field is applied perpendicular to the core wire.

19. An improved method of capturing embolic material contained within fluid flowing through a body vessel; the method comprising the steps of:
providing a magnetically polarizable, filtering device system comprising a source adapted to generate a magnetic field and a filter device; the filter device including:
a hub;
a set of wires coupled to and extending from the hub, the set of wires including a plurality of struts having a predetermined shape and being configured to move between an expanded state for engagement with the body vessel and a collapsed state for filter retrieval and delivery; the struts defining an internal volume in the expanded state in which the embolic material is captured; and
at least one core wire a portion of which is magnetically polarizable; the core wire being coupled to the hub such that the magnetically polarizable portion extends into the internal volume defined by the struts;
delivering the filter device in the collapsed state to a targeted location in the body vessel;
allowing the filter device to move to the expanded state for engagement with the body vessel;
capturing embolic material in the internal volume of the filter device; and
applying an alternating magnetic field to cause the core wire to move or vibrate;
wherein the movement of the core wire break-ups the captured embolic material in order to maintain the flow of fluid through the body vessel.

20. The method of claim 19, wherein the method further comprises the steps of:
allowing the filter device to move from the expanded state to the collapsed state; and
retrieving the filter device from the targeted location in the body vessel.

21. The method of claim 19, wherein the step of applying a magnetic field applies a magnetic field whose strength is predetermined to generate sufficient movement of the core wire to transfer enough force to the embolic material to cause fragmentation thereof.
